(19) United States
(12) Patent Application Publication
(10) Pub. No.: US 2009/0165186 A1
(43) Pub. Date: Jul. 2, 2009

(54) Disposable hospital gown with stethoscope protector/cover

(76) Inventors: Daniel Mijares, Dallas, TX (US); Jamal Lone, Plano, TX (US)

Correspondence Address: Mr. Christopher John Rourk;Jackson Walker LLP, 901 Main Street, Suite 6000, DALLAS, TX, 75202, US

(21) Appl. No.: 12/006,042

(22) Filed: Dec. 31, 2007

(51) Int. Cl. A41D 10/00; A61B 7/02

(52) U.S. Cl. 2/83; 2/114; 181/131

(57) ABSTRACT

A disposable hospital patient's gown with a stethoscope protector or cover for preventing the stethoscope of healthcare provider from being contaminated, so as to prevent transmission of infectious diseases to the patients.

### REFERENCES REGARDING THE NEED FOR A DISPOSABLE HOSPITAL GOWN WITH STETHOSCOPE PROTECTOR/COVER

A prospective study to determine whether cover gowns in addition to gloves decrease nosocomial transmission of vancomycin-resistant enterococci in an intensive care unit.
Infect Control Hosp Epidemiol. 2002 August; 23(8):424-8.
Srinivasan A, Song X, Ross T, Merz W, Brower R, Perl T M.

To gown or not to gown: the effect on acquisition of vancomycin-resistant enterococci.
Clin Infect Dis. 2002 Jul. 1; 35(1):18-25. Epub 2002 Jun. 7.
Puzniak L A, Leet T, Mayfield J, Kollef M, Mundy L M.

Reducing wound infections. Improved gown and drape barrier performance
J A Moylan, K T Fitzpatrick, K E Davenport—Archives of Surgery, 1987—Am Med Assoc.

The role of gown and glove isolation and strict handwashing in the reduction of nosocomial infection in children with solid organ transplantation.
Pediatric Critical Care, Critical Care Medicine. 29(2):405-412, February 2001.
Slota, Margaret R N, M N; Green, Michael M D, M P H; Farley, Adrianne R N; Janosky, Janine PhD; Carcillo, Joseph M D
Stethoscopes as possible of infection by staphylococci. Breathnach, A S, et al. Br Med J. 1992; 305:1573-1574.
Stethoscopes: a potential source of nosocomial infections? Jones, J S, et al. Ann Energ Med. 1992; 26:296-299.
Contaminated Stethoscopes Revisited. Melinda A. Smith, M P H; John J. Mathewson, PhD; I.
Alan Uler, M D; Ernesto G. Scerpella, M D; and Charles D. Ericsson, M D; Arch Intern Med/vol. 156, Jan. 8, 1996, pp. 82-84.
The Stethoscope. A Potential Source of Nosocomial Infection? Mark A. Marinella, M D; Carl Pierson, PhD; and Carol Chenoweth, M D; Arch Intern Med/vol. 157, Apr. 14, 1997, pp. 786-790.
Graham-Field To Distribute Patented Stethoscope Cover; Scope Shield Protects Patients Against Infectious Diseases, Graham-Field Health Products; Inc., Feb. 18, 1998.
Bacterial contamination of hospital physician stethoscopes. Bernard L, et al. Infect Control Hosp Epidemiol. 1999; 20: 626-628.
Stethoscopes and nosocomial infection. Sengupta S, e al. Indian J Peiatr. 2000. 67(3): 197-199.
Contamination of gowns, gloves and stethoscopes with Vancomycin [Vancocin] resistant enterococci. Zachary K, et al. Infect Control Hosp Epidemiol. 2001. 22: 560-563.
Stethoscope contamination in the neonatal intensive care unit. Wright, I M; Orr, H; Porter, C. J Hosp Infect. 1995; 29(1): 65-8.

### FEDERALLY SPONSORED RESEARCH

N/A

### SEQUENCE LISTING OR PROGRAM

N/A

### BACKGROUND OF THE INVENTION

1. Field of Invention

This invention relates to medical stethoscopes and disposable hospital gowns. The primary mode of isolation in hospitals is barrier protection. Gloves and gowns are the most commonly used barrier to prevent bacterial cross contamination.

Even though gowns are effective if used correctly, the need for frequent patient monitoring results in breaks in barrier protection. For example, the use of a stethoscope is a well-documented defect in isolation techniques. The stethoscope, typically a personal device owned by the health care professional, is used to assess the chest, heart and abdomen of every patient by multiple health care professionals.

In the conventional use of the stethoscope by physicians, nurses and other health personnel, the stethoscope is not sterilized between the examinations of patients. Typically, as the stethoscope is directly applied to the skin, the diaphragm, body of the stethoscope, and healthcare workers hands will become contaminated and has the potential of contaminating the next patient. The present invention is a system of not only reducing transmission of infectious organisms, but also a means to improve hygiene by reducing transmission of body fluids, secretions, topical medications and other biohazards. It allows the physician to use his own, high quality stethoscope for the examination of his patient without causing the stethoscope to act as a fomitic device.

Disposable gowns combined with single use stethoscopes have been proposed as a solution to eliminate breaks in barrier protection, but these stethoscopes are often left in the room of patients carrying resistant organisms and the supply quickly becomes contaminated. More importantly, the single-use stethoscope is hardly ever used in place of the health care professional's personal stethoscope. Also, due to that fact that several health care providers use them the method is un-appealing and unhygienic. As a result, the single-use stethoscope has not been effective in infection control isolation and control.

As efforts to clean stethoscopes with devices such as disinfectant wipes, which are not completely effective, can cause skin irritation and the use is extremely infrequent, there have been a number of other attempts to develop an effective and economical solution to the problem of micro-organism transmission via the stethoscope.

The present invention is a complete isolation system of not only reducing transmission of infectious organisms, but also a means to improve hygiene by reducing transmission of body fluids, secretions, topical medications and other biohazards via the stethoscope. And since the stethoscope cover is part of the hospital gown, it is readily available.

Resistant infectious organisms affect hospitalized patients at an ever increasing rate. Outbreaks of resistant bacteria are now also commonly reported in the non-hospitalized general population. Patients are frequently exposed to these organisms and other biohazards because of inadequate isolation techniques which do not maintain sufficient barriers between patients and their health care providers. Breaks in technique result in cross contamination of previously uninfected patients, which in turn, causes increased colonization and infection with resistant organisms. These infections cost millions of dollars annually and add to patient morbidity and hospital length of stay.

Our goal is to provide a high level of hygiene and protection from multi-resistant organisms to the general population during its encounter with the healthcare system. These gowns/stethoscope protectors should be implemented not only in the hospitals but also in the doctor's office, veterinarian's offices, nursing homes, etc.

2. Prior Art

There is no prior art on a disposable hospital gown with stethoscope protector/cover.

### CROSS-REFERENCE TO RELATED APPLICATIONS

### U.S. Patent Documents Regarding Stethoscope Covers

| | | |
|---|---|---|
| 4401125 | August 1983 | Taylor et al. |
| 4461368 | July 1984 | Plourde |
| 4867268 | September 1989 | Ulert |
| 4871046 | October 1989 | Turner |
| 5424495 | June 1995 | Wurzburger |
| 5448025 | September 1995 | Stark et al. |
| 5466898 | November, 1995 | Gilbert |
| 5528004 | June 1996 | Wurzburger |
| 5587561 | December 1996 | Budayr et al. |
| 5686706 | November 1997 | Wurzburger |
| 5808244 | September 1998 | Knight et al. |
| 6006856 | December, 1999 | Skubal |
| 6,009,971 | Jan. 4, 2000 | Weidman, et al. |
| 6186957 | February, 2001 | Milam |
| 6468611 | October, 2002 | Haskin |
| 6649236 | November, 2003 | Haskin |
| 7117971 | October 2006 | Cornacchia |

### U.S. Patent Documents Regarding Disposable Hospital Gowns

| | | |
|---|---|---|
| 3824625 | July 1974 | Green |
| D236293 | August 1975 | Banks |
| 3911499 | October 1975 | Benevento et al. |
| 4000521 | January 1977 | Zoephel et al. |
| 4017909 | April 1977 | Brandriff |
| 4052752 | October 1977 | Green |
| 4075716 | February 1978 | Collins |
| 4408357 | October 1983 | Toth |
| 4504977 | March 1985 | King et al. |
| 4504978 | March 1985 | Gregory et al. |
| 4507978 | April 1985 | Tanaka et al. |
| 4586196 | May 1986 | White |
| 4608719 | September 1986 | Lunt |
| 4819275 | April 1989 | Lunt |
| D356204 | March 1995 | Derrickson |
| 5414867 | May 1995 | Bowling et al. |
| 6054091 | April 2000 | Miller et al. |
| 6378136 | April 2002 | Matsushita |
| 2001/0032346 | October 2001 | Matsushita et al. |

U.S. Pat. Nos. 4,867,265, 5,486,659, 5,564,431, 5,747,751, and 5,813,992 disclose a cover which only extends over the head of the stethoscope. As a result, the rest of the stethoscope remains exposed and susceptible to contamination. It is not attached to an isolation gown.

U.S. Pat. No. 5,623,131 discloses a two piece stethoscope cover that has a first piece which covers the head and a second piece that covers the tube that connects the head to the ear tubes. This two piece cover is impractical and therefore not applicable in the hospital or office setting where the nurse or doctor has to see multiple patients in a short time interval. Also, this does not encompass the use of an isolation gown.

U.S. Pat. No. 5,592,946 discloses a stethoscope cover that leaves the ear tubes and the head exposed. Furthermore, this cover is made of a hypoallergenic fabric which may not be a barrier to biohazards and the only disclosed functions of the cover is to prevent allergic reactions of the wear and to decorate the stethoscope to help distract anxious patients.

U.S. Pat. No. 5,466,898 discloses a stethoscope isolation system which, although completely covers the head, leaves most of the ear tubes exposed. The use of the spreader attachment is very impractical. Also, the hard proximal part of the stethoscope sleeve makes it heavier, less practical, and more expensive to produce than our current invention. No mention of an isolation gown is made.

U.S. Design Pat. No. 376,043 shows a stethoscope cover which does extend up the length of the ear tubes. This cover does not protect the side of the head of the stethoscope that contacts the patient, and, the shown design would be awkward to use. This does not involve the use of an isolation gown in conjunction with the stethoscope protector.

U.S. Pat. Nos. 5,424,495, 5,528,004 and U.S. Pat. No. 5,686,706, each issued to Wurzberger disclose a dispensable, disposable cover for stethoscopes. The dispensable, disposable cover set forth therein prevents the transfer of disease or other contaminants through the incorporation of a shield having an adhesive backing for removably attaching the shield over the entire surface area of a diaphragm of the stethoscope. The shield is peelably detachable from the stethoscope diaphragm after use. In various embodiments of the cover set forth therein, the shield includes a pull-tab or flap which allows for the ease in manipulating the shield. However, these shields are individually dispensed from a dispenser by the health care professional and applied to the stethoscope individually. This requires the health care professional to come into unnecessary contact with the shield when applying the shield to the stethoscope. Moreover, this requires that a dispenser containing a supply of stethoscope shields be either carried by the health care professional or the need for a dispenser to be placed in not only each room but also corridors and waiting rooms within the medical facility. Moreover, should such dispenser not be readily available to the health care professional, the likelihood of such a shield being used is minimal.

Similarly, U.S. Pat. No. 5,448,025 issued to Stark et al. discloses a shield for temporary securement to a stethoscope head to completely cover a diaphragm of the stethoscope head and seal the interior and diaphragm from the ambient atmosphere. The cover includes a flat, double-sided thin plastic sheet and a layer of adhesive on one side of the sheet to releasably secure the cover to the outer rim surface of the stethoscope head. The shield is accommodated on a tape having a plurality of such shields thereon which is withdrawn from a dispenser, removed from the tape and applied to the stethoscope. Again, it is necessary for the health care professional to handle the shield and the dispenser in order to remove the shield from the tape and apply the shield to the stethoscope. Additionally, as discussed hereinabove, such a dispensing system requires that the health care professional carry a supply of the shields with them or that a dispensing mechanism be provided within each room of a medical facility. Clearly, there will be times when it is impractical to use the shield because such shield is not readily available to the health care professional. No mention is made in relation to an isolation gown.

A still further effort to overcome the aforementioned shortcomings, the stethoscope shield set forth in U.S. Pat. No. 5,587,561 issued to Budayr et al. includes an annular flange disposed to extend generally upwardly from the shield towards the head of the stethoscope to protect the stethoscope from body fluids disposed on those patient's skin. However, the shields are provided in a stack with the uppermost diaphragm being removed from the stack and applied to the stethoscope in the manner discussed hereinabove. Once again, such a system requires handling of the shield in some manner and likewise requires the medical care professional to carry a dispenser along with them or have a dispenser provided in each room of a medical facility which, as noted hereinabove, is not feasible. Additionally, this system requires the exposure of the adhesive to the environment over long periods of time and the use of two hands during application.

U.S. Pat. No. 3,092,252 to Brause. et al. discloses packages with folds at one end for use in protecting humans from pathogens that otherwise might be present on thermometers, tongue depressors, surgical instruments, fingers/hands, etc. The inner surface of the packages of this invention initially are sterile, though the objects placed within the packaging are not sterile. Multiple folds are utilized in the practice of this invention. Complicated folding is utilized in the packaging of this invention.

U.S. Pat. No. 3,215,265 to Welin-Berger discloses the use of a plurality of sheaths to protect patients from cross-contamination from thermometers. An inner package covers a thermometer, and may remain about the thermometer during insertion into a body orifice for temperature measurement. An outer package contains the inner package along with its contents.

U.S. Pat. No. 6,186,957 to Milam discloses a stethoscope isolation system that covers the head, body and ear tubes of the stethoscope. This is impractical as the proximal ends terminate in the ear piece, making the cover difficult to put on and take off. It also called for the use of a bendable metal fastener, which would further add to the difficulty with the use of the cover. As such, the design would be awkward and impractical. No mention of it in relation to an isolation gown is made.

U.S. Pat. No. 3,847,280 to Poncy discloses a sterile package for clinical thermometers and similar diagnostic equipment elements, where packaging may be stripped from sheathing surrounding the thermometer or other diagnostic equipment elements. Multiple layers and coatings are used, including ones for lubricants. One embodiment utilizes a plurality of packages that are attached in succession and separated by perforated sections for convenient separation.

U.S. Pat. No. 5,365,023 to Lawton discloses an elastic latex, disk-shaped cover for fitting over various stethoscope heads. As a result, the rest of the stethoscope remains exposed and susceptible to contamination.

U.S. Pat. No. 5,446,897 to Ross. et al. discloses a dispenser apparatus for dispensing disposable stethoscope diaphragms. The apparatus is designed to prevent the spread of infections.

U.S. Pat. No. 5,564,431 to Seward discloses a flexible stethoscope cover and dispensing/packaging system for the head of a stethoscope. The cover basically is a rolled tube that is unrolled to cover a stethoscope head, much in the manner that a condom is applied. As a result, the rest of the stethoscope remains exposed and susceptible to contamination.

Thus, there exists a need for improved methods for preventing the transmission of infectious organisms between patients when a stethoscope is used, and the present invention provides a solution to this problem.

### SUMMARY

This invention relates to medical stethoscopes and disposable hospital gowns. The present invention is a complete isolation system. It provides a means of not only reducing transmission of infectious organisms, but also a means to improve hygiene by reducing transmission of body fluids, secretions, topical medications and other biohazards between the healthcare provider and the patient. This is done by implementing a barrier method to cover the healthcare provider and his stethoscope at the same time.

In the conventional use of the stethoscope by physicians, nurses and other health personnel, the stethoscope is not sterilized between examinations of patients. Typically, as the stethoscope is directly applied to the skin, the diaphragm and body will become contaminated and has the potential of contaminating the next patient. The present invention is a system of not only reducing transmission of infectious organisms, but also a means to improve hygiene by reducing transmission of body fluids, secretions, topical medications and other biohazards. It allows the physician to use his own, high quality stethoscope for the examination of his patient without causing the stethoscope to act as a fomitic device.

Resistant infectious organisms affect hospitalized patients at an ever increasing rate. Patients are frequently exposed to these organisms and other biohazards because of inadequate isolation techniques which do not maintain sufficient barriers between patients and their health care providers. Breaks in technique result in cross contamination of previously uninfected patients, which in turn, causes increased colonization and infection with resistant organisms. These infections cost millions of dollars annually and add to patient morbidity and hospital length of stay.

The primary mode of isolation in hospitals is barrier protection. Gloves are the most commonly used barrier to prevent bacterial cross contamination. Even though gloves are effective if used correctly, the need for frequent patient monitoring results in breaks in barrier protection. For example, the use of a stethoscope is a well-documented defect in isolation techniques. The stethoscope, typically a personal device owned by the health care professional, is used to assess the chest and heart of every patient by multiple health care professionals. The stethoscope is carried by each health care professional from patient to patient and acts as a fomite, increasing organism transmission. There are numerous articles documenting the harboring of organisms by the stethoscope, and the manner by which these organisms are transmitted to others.

Single-use stethoscopes have been implemented as a solution to eliminate breaks in barrier protection, but these stethoscopes are often left in the room of patients carrying resistant organisms and the supply quickly becomes contaminated. More importantly, the single-use stethoscope is hardly ever used in place of the health care professional's personal stethoscope. As a result, the single-use stethoscope has not been effective in infection control isolation and control.

### CONCLUSION, RAMIFICATION AND SCOPE OF INVENTION

The present invention is intended to make use isolation gowns and uncontaminated stethoscopes as quick and easy as possible and to encourage their use by the busy healthcare providers by making the stethoscope protector a part of the gown. This will result in reduction of spread of disease and improvement in hygiene, ultimately saving patient lives and taxpayer money spent on healthcare.

The invention provides not only an uncontaminated diaphragm surface for each examination, but also protects all other parts of the stethoscope that usually get in contact with the patient. While the previous inventions such as the removable diaphragm covers cover the diaphragm they lack coverage of other areas of the stethoscope that come into contact with patient and healthcare provider. The Stethoscope Cover by Milam or Gilbert provides the desired hygienic condition of the stethoscope, but lack ease of quick interchange and covers parts of the stethoscope that never get into contact with the patient making them impractical. Although it is over six years since the Milam's device was invented and over 12 years since Gilbert's device was invented, they apparently have not found their use in medical practice. The invention also is attached to an isolation gown, providing a complete isolation system, which none of the previous patents have mentioned.

The present invention fits all conventional stethoscopes and provides all the advantages described at length in previous patents concerning stethoscope protectors, while being user friendly.

### DRAWINGS

### Brief Description of the Drawings

Preferred features of the present invention are disclosed in the accompanying drawings, wherein similar reference characters denote similar elements throughout the several views, and wherein:

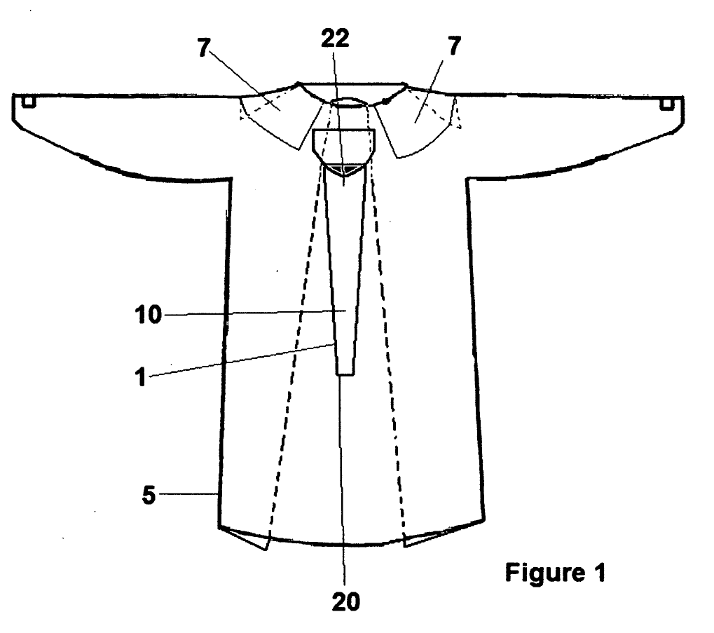

FIG. 1 shows a front view of the isolation gown with the stethoscope cover;

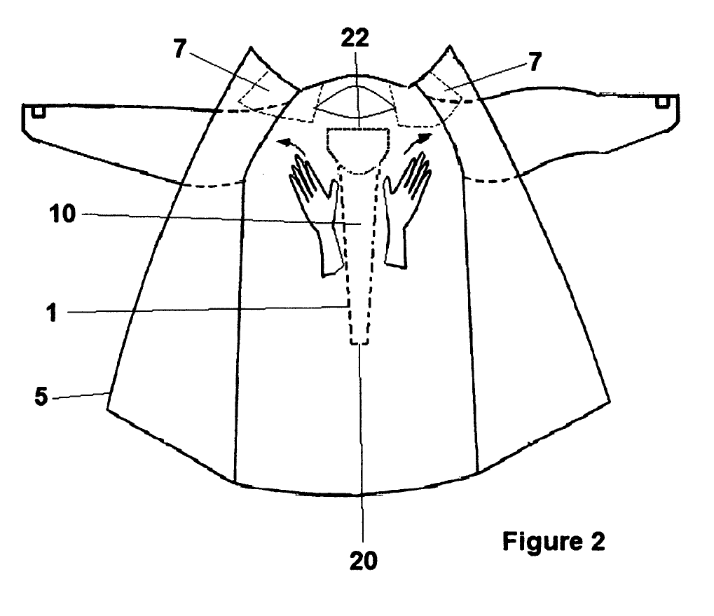

FIG. 2 shows a back view of the isolation gown with the stethoscope protector;

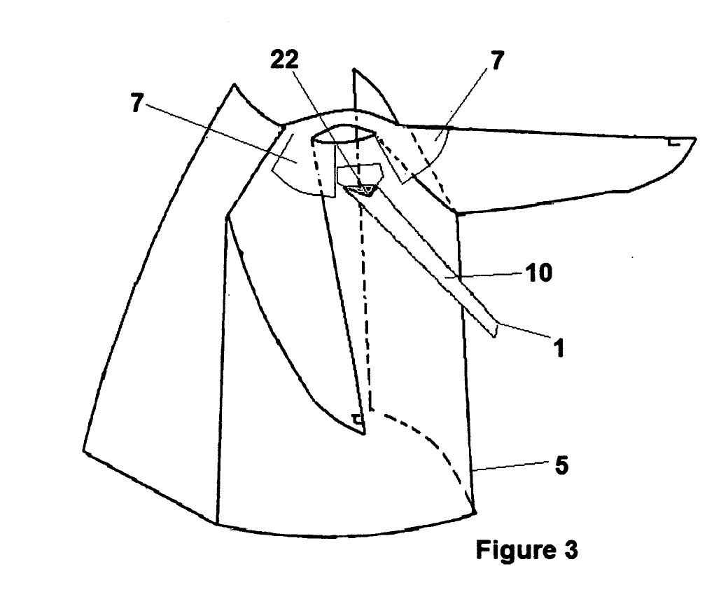

FIG. 3 shows a side view of the isolation gown with the stethoscope protector;

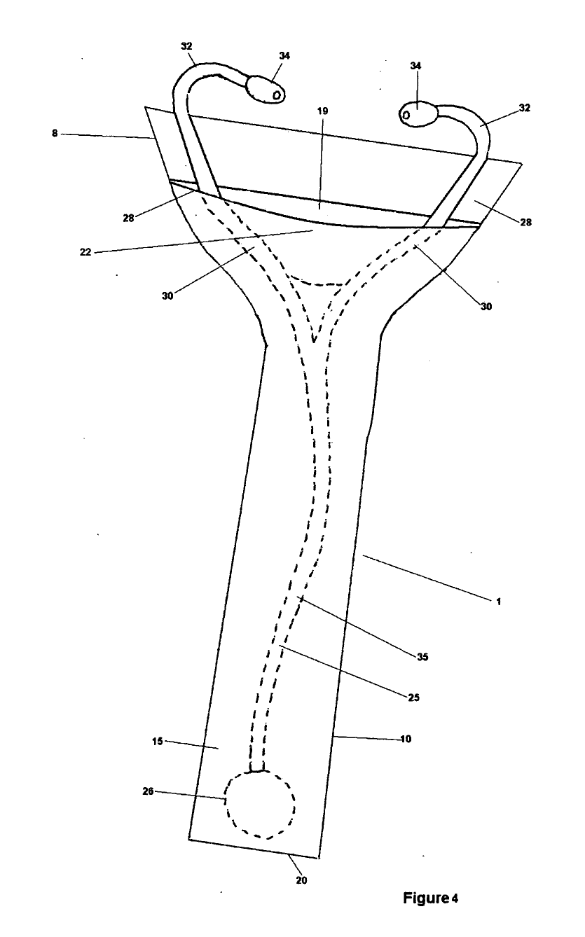

FIG. 4 shows a front view of a stethoscope cover according to the present invention with a stethoscope inserted in the cover;

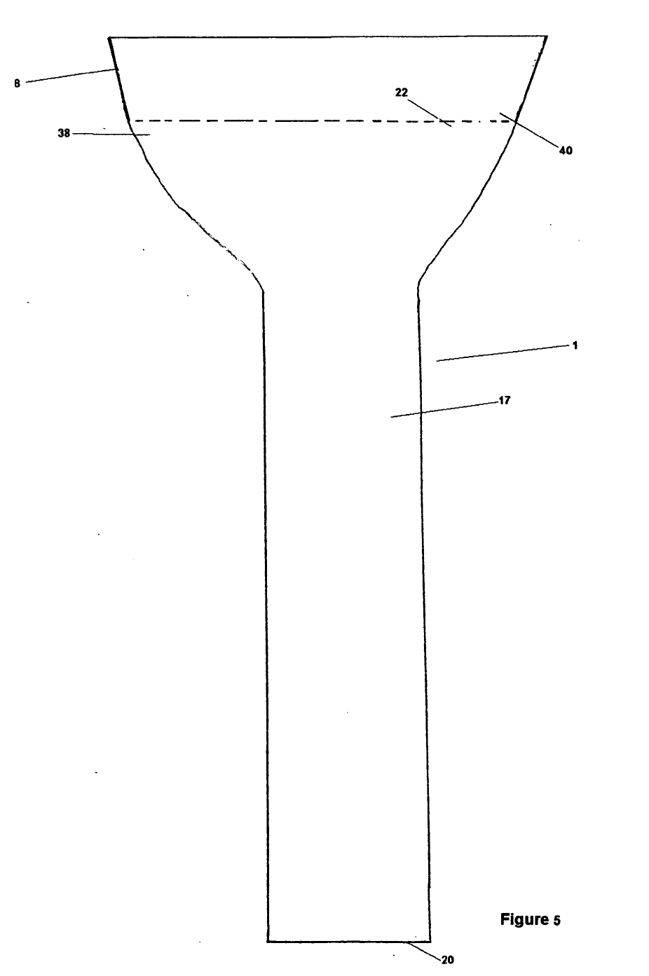

FIG. 5 shows a back view of the stethoscope cover of FIG. 4 with the stethoscope removed;

### DETAILED DESCRIPTION OF THE PREFERRED EMBODIMENTS

Referring to the figures, a stethoscope cover 1 according to the present invention includes a body 10 having a front panel 15 and a back panel 17. Front and back panels 15, 17 are joined to form a pouch 19 with a closed distal end 20 and an open proximal end 22. Pouch 19 is sized to receive a portion of a stethoscope 25. Specifically, stethoscope 25 has a head 26 for placing on a patient and acquiring sound, first and second ear tubes 28, each with a distal end 30 and a proximal end 32 terminating in an ear piece 34 insertable in the ear to hear the sound, and a connector section 35 connecting distal ends 30 of first and second ear tubes 28 to head 26. Pouch 19 is configured and dimensioned to receive head 26, connector section 36, distal end 30, and a portion of the first and second ear tubes 28 of stethoscope 25. Portion 8 is a mechanism of attachment between the gown and stethoscope protector.

As cover 1 and isolation gown 5 are made of a viral, bacterial, and fluid impermeable material, cover 1 prevents stethoscope 25 from being contaminated with these and other biohazards. Furthermore, the material that cover 1 and isolation gown 5 are made of is also acoustically transmitting to minimize interference with the normal operation of stethoscope 25. Examples of such a material include a thin, flexible polymeric material such as polyethylene, latex rubber, silicone, soft vinyl, urethane, cellophane, and the like. These are the same materials that are used for gloves and other protective garments and are well known in the art. There are a number of ways that front and back panels 15, 17, a continuous sheet, or multiple sheets can be joined together to form pouch 19 so that a tight seal between the two is formed. For example, front and back panels 15, 17 can be welded or glued at their edges.

In use, a health care professional having a clean stethoscope hanging from his neck takes a stethoscope cover 1 and isolation gown 5 from a supply of isolation gowns with attached stethoscope protective covers. In order to make the isolation gowns and stethoscope protective covers readily accessible and to increase user compliance, the supply can be conveniently located, e.g. near a box of examination gloves on an isolation cart. Isolation gown 5 is placed on and cover 1 is slid over the stethoscope until all of head 26, connector section 36, and a portion of ear tubes 28 are within pouch 19. As both cover 1 and stethoscope 25 are clean, it does not matter if the health care professional dons gloves before or after cover 1 is placed on stethoscope 25. For optimal results, gloved hands should be used to grasp the attached isolation wings 7, which are used to prevent contact with contaminants to the stethoscope when placing the device on or taking it off.

Once ear pieces 34 are inserted in the ears, the patient can be examined. Stethoscopes can be provided with a head that amplifies sound, a diaphragm on a ring responsive to sound, or a combination bell/diaphragm in which the mode of use is selected with a valve. Cover 1 is made of a material such that tactile feel of stethoscope 25 through cover 1 would be sufficient for use and manipulation of stethoscope 25, making either of the panels or a portion of the panels of a translucent or transparent material would be desirable to visualize a valve of a combination head. Furthermore, pouch 19 is wide enough to allow head 26 to spin around so that the bell or diaphragm can be appropriately positioned through cover 1.

After examination of the patient, ear pieces 34 are removed from the ears and cover 1 is pulled down off of stethoscope 25 using the isolation wings 7. The used cover 1 can be discarded along with the isolation gown and contaminated gloves.

While various descriptions of the present invention are described above, it should be understood that the various features can be used singly or in any combination thereof. Therefore, this invention is not to be limited to only the specifically preferred embodiments depicted herein.

Further, it should be understood that variations and modifications within the spirit and scope of the invention may occur to those skilled in the art to which the invention pertains. Accordingly, all expedient modifications readily attainable by one versed in the art from the disclosure set forth herein that are within the scope and spirit of the present invention are to be included as further embodiments of the present invention.

1-2. (canceled)

3. A disposable gown, comprising:
a body portion having sleeves attached thereto, the body portion having a front and a back;
a defined neck opening of the body portion;
a stethoscope protector coupled to the body portion proximate to the defined neck opening;
a fastener configured to allow the user to remove disposable gown without manually removing the stethoscope from the stethoscope protector; and
wherein the disposable gown is configured to allow a user to enter the disposable gown while wearing a stethoscope and to place the stethoscope into the stethoscope protector.

4. The disposable gown of claim 3 wherein the stethoscope protector is coupled to the front of the body portion.

5. The disposable gown of claim 3 wherein the body portion is coated with a polymeric film from the group comprising polyurethane, elastomeric polyester, blends of polyurethane and polyester, and styrene/butadiene block copolymers.

6. The disposable gown of claim 3 wherein the stethoscope protector is coated with a polymeric film from the group comprising polyurethane, elastomeric polyester, blends of polyurethane and polyester, and styrene/butadiene block copolymers.

7. The disposable gown of claim 3 wherein the stethoscope protector further comprises an elongated pouch with a closed distal end and an open proximal end.

8. The disposable gown of claim 7 wherein the elongated pouch is configured and dimensioned to receive a head and a tubing portion of the stethoscope.

9. A method for using a disposable gown having an attached stethoscope protector comprising:
inserting arms of a user through sleeves of the disposable gown;
fastening the disposable gown using a fastener so as to secure the disposable gown on the user;
inserting a stethoscope into the attached stethoscope protector to allow the user to use the stethoscope, wherein the stethoscope protector provides a barrier between the stethoscope and a patient.

10. The method of claim 9 wherein the fastener is a quick-release fastener, and further comprising removing the disposable gown by pulling the disposable gown away from the user so as to release the quick-release fastener and remove the stethoscope from the attached stethoscope protector in a single action.

11. A disposable gown, comprising:
a body portion having sleeves attached thereto, the body portion having a front and a back;
a defined neck opening of the body portion;
means for fastening the body portion; and
means for protecting a stethoscope.

12. The disposable gown of claim 11 wherein the means for protecting the stethoscope is coupled to the front of the body portion.

13. The disposable gown of claim 11 wherein the body portion is coated with a polymeric film from the group comprising polyurethane, elastomeric polyester, blends of polyurethane and polyester, and styrene/butadiene block copolymers.

14. The disposable gown of claim 11 wherein the means for protecting the stethoscope is coated with a polymeric film from the group comprising polyurethane, elastomeric polyester, blends of polyurethane and polyester, and styrene/butadiene block copolymers.

15. The disposable gown of claim 11 wherein the means for protecting the stethoscope further comprises an elongated pouch with a closed distal end and an open proximal end.

16. The disposable gown of claim 15 wherein the elongated pouch is configured and dimensioned to receive a head and a tubing portion of the stethoscope.

17. The disposable gown of claim 11 wherein the means for fastening the body portion comprises an adhesive fastener.

18. The disposable gown of claim 20 wherein the adhesive fastener is configured to allow the user to remove disposable gown without manually removing the stethoscope from the stethoscope protector.
